(19) United States
(12) Patent Application Publication
(10) Pub. No.: US 2024/0132623 A1
(43) Pub. Date: Apr. 25, 2024

(54) Methods for Treating Multiple Myeloma

(71) Applicant: Celgene Corporation, Summit, NJ (US)

(72) Inventors: Nicholas Stong, Hasbrouck Heights, NJ (US); Patrick R. Hagner, Sparta, NJ (US); William Edward Pierceall, Madison, NJ (US); Anjan Thakurta, Basking Ridge, NJ (US); Jennifer Erin Flynt, Hattiesburg, MS (US); Maria Ortiz-Estevez, Donostia (ES)

(73) Assignee: Celgene Corporation, Summit, NJ (US)

(21) Appl. No.: 18/546,155

(22) PCT Filed: Feb. 25, 2022
(86) PCT No.: PCT/US2022/017844

§ 371 Date: Aug. 10, 2023

Related U.S. Application Data
(60) Provisional application No. 63/154,104, filed on Feb. 26, 2021.

(51) Int. Cl. C07K 16/40; A61K 45/06; C12Q 1/6886

(52) U.S. Cl. CPC C07K 16/40; A61K 45/06; C12Q 1/6886; C12Q 2600/156

(57) ABSTRACT

Provided herein are methods of treating multiple myeloma, comprising administering to a subject with multiple myeloma an inhibitor of Nuclear Receptor Binding SET Domain Protein 2 (NSD2). Also provided are methods of use wherein the multiple myeloma has previously been determined to have a 4:14 chromosome translocation (t(4;14)).

### I. CROSS-REFERENCE TO RELATED APPLICATIONS

This application claims the benefit of priority of U.S. Provisional Application No. 63/154,104, filed Feb. 26, 2021, which is incorporated by reference herein in its entirety for any purpose.

### II. FIELD

Provided herein are methods of treating multiple myeloma, comprising administering to a subject with multiple myeloma an inhibitor of Nuclear Receptor Binding SET Domain Protein 2 (NSD2). Also provided are methods of treatment wherein the multiple myeloma has previously been determined to have a 4:14 chromosome translocation (t(4;14)).

### III. BACKGROUND

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin, except in some patients (estimated at 1% to 5%) whose myeloma cells do not secrete these proteins (termed non-secretory myeloma). M protein, short for monoclonal protein, also known as paraprotein, is a particularly abnormal protein produced by the myeloma plasma cells and can be found in the blood or urine of almost all patients with multiple myeloma, except for patients who have non-secretory myeloma or whose myeloma cells produce immunoglobulin light chains with heavy chain.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Cytogenetics is an important prognostic marker in multiple myeloma. The approximately fifteen percent of newly diagnosed MM patients with the t(4;14) chromosome translocation demonstrate poor prognosis, including short progression free survival (PFS) and overall survival (OS), which is only partially mitigated by existing therapies. Accordingly, specific therapeutic strategies for this subpopulation are greatly needed. Ortiz et al., Blood (2019) 134 (Supplement_1): 366.

### IV. SUMMARY

Provided herein are methods of treating multiple myeloma, comprising administering to a subject with multiple myeloma an inhibitor of Nuclear Receptor Binding SET Domain Protein 2 (NSD2).

Also provided are methods of use wherein the multiple myeloma has previously been determined to have a 4:14 chromosome translocation (t(4;14)).

The following non-limiting embodiments are provided.

Embodiment 1 is a method of treating multiple myeloma, comprising administering to a subject with multiple myeloma a therapeutically effective amount of an inhibitor of Nuclear Receptor Binding SET Domain Protein 2 (NSD2).

Embodiment 2 is the method of embodiment 1, wherein the multiple myeloma has previously been determined to have a 4:14 chromosome translocation (t(4;14)).

Embodiment 3 is the method of embodiment 2, wherein the t(4;14) results in a disruption in the NSD2 gene.

Embodiment 4 is the method of embodiment 3, wherein the disruption in the NSD2 gene is located after the transcription start site of NSD2.

Embodiment 5 is the method of embodiment 3 or embodiment 4, wherein the disruption in the NSD2 gene is located after the translation start site of NSD2.

Embodiment 6 is the method of any one of embodiments 3-5, wherein the disruption in the NSD2 gene is located before the translation stop site of NSD2.

Embodiment 7 is the method of any one of embodiments 3-5, wherein the disruption in the NSD2 gene is located before the first coding exon of the NSD2 gene.

Embodiment 8 is the method of any one of embodiments 3-6, wherein the disruption in the NSD2 gene is located in the first coding exon of the NSD2 gene.

Embodiment 9 is the method of any one of embodiments 3-6, wherein the disruption in the NSD2 gene is located between the start of the first coding exon and the start of the second coding exon of the NSD2 gene.

Embodiment 10 is the method of any one of embodiments 3-9, wherein the disruption in the NSD2 gene is located at or after genomic position 1,871,393 of Genome Reference Consortium Human Build 38 patch release 13 (GRCh38.p13).

Embodiment 11 is the method of any one of embodiments 3-10, wherein the disruption in the NSD2 gene is located between genomic position 1,871,393 and genomic position 1,900,655 of GRCh38.p13.

Embodiment 12 is the method of any one of embodiments 3-10, wherein the disruption in the NSD2 gene is located at or after genomic position 1,900,655 of GRCh38.p13.

Embodiment 13 is the method of any one of embodiments 3-10, wherein the t(4;14) is located between genomic position 1,900,655 and genomic position 1,982,207 of GRCh38.p13.

Embodiment 14 is the method of any one of the preceding embodiments, wherein the multiple myeloma expresses a truncated NSD2 protein.

Embodiment 15 is the method of any one of embodiments 1-13, wherein the multiple myeloma expresses a full-length NSD2 protein.

Embodiment 16 is the method of embodiment 15, wherein the multiple myeloma expresses elevated levels of the full-length NSD2 protein.

Embodiment 17 is the method of any one of embodiments 2-16, wherein the 4:14 chromosome translocation (t(4;14)) was identified by a method comprising in situ hybridization, PCR, RT-PCR, RNA sequencing, fluorescence in situ hybridization (FISH), transcript in situ hybridization, whole genome sequencing, whole exome sequencing, mixed ligation probe assays, mass spectrometry, and/or MALDI-TOF.

Embodiment 18 is the method of any one of the previous embodiments, wherein the inhibitor of NSD2 is selected from an antibody, a small molecule, an aptamer, an siRNA, and an antisense oligonucleotide.

Embodiment 19 is the method of any one of the previous embodiments, wherein the method comprises administering at least one second therapeutic agent.

Embodiment 20 is the method of embodiment 19, wherein at least one second therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, and a nuclear export inhibitor.

Embodiment 21 is the method of embodiment 19, wherein at least one second therapeutic agent is selected from lenalidomide, thalidomide, pomalidomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

Embodiment 22 is the method of embodiment 19, wherein the at least one second therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl)piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.

Embodiment 23 is a method of selecting a subject with multiple myeloma for treatment with an NSD2 inhibitor, comprising determining whether the subject has a 4:14 chromosome translocation (t(4;14)), wherein if the subject has a t(4;14), the subject is selected for treatment with an NSD2 inhibitor.

Embodiment 24 is a method of predicting whether a subject with multiple myeloma will benefit from treatment with an NSD2 inhibitor, comprising determining whether the subject has a 4:14 chromosome translocation (t(4;14)), wherein if the subject has a t(4;14) translocation, the subject is predicted to benefit from treatment with an NSD2 inhibitor.

Embodiment 25 is the method of embodiment 23 or embodiment 24, wherein the t(4;14) results in a disruption in the NSD2 gene.

Embodiment 26 is the method of embodiment 25, wherein the disruption in the NSD2 gene is located after the transcription start site of NSD2.

Embodiment 27 is the method of embodiment 25 or embodiment 26, wherein the disruption in the NSD2 gene is located after the translation start site of NSD2.

Embodiment 28 is the method of any one of embodiments 25-27, wherein the disruption in the NSD2 gene is located before the translation stop site of NSD2.

Embodiment 29 is the method of any one of embodiments 25-28, wherein the disruption in the NSD2 gene is located before the first coding exon of the NSD2 gene.

Embodiment 30 is the method of any one of embodiments 25-28, wherein the disruption in the NSD2 gene is located in the first coding exon of the NSD2 gene.

Embodiment 31 is the method of any one of embodiments 25-28, wherein the disruption in the NSD2 gene is located between the start of the first coding exon and the start of the second coding exon of the NSD2 gene.

Embodiment 32 is the method of any one of embodiments 25-31, wherein the disruption in the NSD2 gene is located at or after genomic position 1,871,393 of Genome Reference Consortium Human Build 38 patch release 13 (GRCh38.p13).

Embodiment 33 is the method of any one of embodiments 25-32, wherein the disruption in the NSD2 gene is located between genomic position 1,871,393 and genomic position 1,900,655 of GRCh38.p13.

Embodiment 34 is the method of any one of embodiments 25-32, wherein the disruption in the NSD2 gene is located at or after genomic position 1,900,655 of GRCh38.p13.

Embodiment 35 is the method of any one of embodiments 25-32, wherein the t(4;14) is located between genomic position 1,900,655 and genomic position 1,982,207 of GRCh38.p13.

Embodiment 36 is the method of any one of embodiments 23-35, wherein the multiple myeloma expresses a truncated NSD2 protein.

Embodiment 37 is the method of any one of embodiments 23-35, wherein the multiple myeloma expresses a full-length NSD2 protein.

Embodiment 38 is the method of embodiment 37, wherein the multiple myeloma expresses elevated levels of the full-length NSD2 protein.

Embodiment 39 is the method of any one of embodiments 23-38, wherein determining whether the subject has a t(4;14) comprises in situ hybridization, PCR, RT-PCR, RNA sequencing, fluorescence in situ hybridization (FISH), transcript in situ hybridization, whole genome sequencing, whole exome sequencing, mixed ligation probe assays, mass spectrometry, and/or MALDI-TOF.

Embodiment 40 is the method of any one of embodiments 23-39, wherein the method further comprises administering an NSD2 inhibitor.

Embodiment 41 is the method of embodiment 40, wherein the inhibitor of NSD2 is selected from an antibody, small molecule, an aptamer, an siRNA, and an antisense oligonucleotide.

Embodiment 42 is the method of embodiment 40 or embodiment 41, wherein the method comprises administering at least one second therapeutic agent.

Embodiment 43 is the method of embodiment 42, wherein at least one second therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, and a nuclear export inhibitor.

Embodiment 44 is the method of embodiment 42, wherein at least one second therapeutic agent is selected from lenalidomide, thalidomide, pomalidomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

Embodiment 45 is the method of embodiment 42, wherein the at least one second therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl)piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.

### V. BRIEF DESCRIPTION OF THE DRAWINGS

FIG. 1A shows the survival probability of the 329 newly diagnosed multiple myeloma (NDMM) patients over time. FIG. 1B shows a density plot of the overall survival of the 329 NDMM patients. FIG. 1C shows identification and grouping according to patients having overall survival of less than 24 months.

FIG. 2A shows that translocations that disrupt the coding of the NSD2 protein correlate with shorter overall survival. FIG. 2B shows patients separated into three groups: no disruption, early disruption, and late disruption, with each group having an increasingly poor prognosis in terms of time to overall survival.

FIG. 3A shows that analysis of the RNA-seq data identified three groups of patients: (1) the full coding fusion transcript group, where there is little to no DNA disruption, (2) the truncated fusion transcript group characterized by late DNA disruption, and (3) a small subset of patients having no measured fusion transcript expression. FIG. 3B shows that patients expressing the truncated fusion protein have a worse overall survival than patients expressing the full coding fusion transcript or no fusion transcript.

### VI. DETAILED DESCRIPTION OF THE INVENTION

### A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of.” Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C.” An exception to this definition will occur only when a combination of elements, functions, steps or acts are in some way inherently mutually exclusive.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein the term “Genome Reference Consortium Human Build 38 patch release 13” abbreviated “GRCh38.p13” or “hg38” refers to the human reference genome assembly of that name, dated Feb. 28, 2019. The GenBank accession for GRCh38 is GCA_000001405.28, also available in RefSeq under accession GCF_000001405.39

As used herein the terms “NSD2” and “nuclear receptor binding SET domain protein 2” are used interchangeably to refer to an NSD2 polypeptide or a nucleic acid, such as a gene, encoding the polypeptide. An NSD2 polypeptide is a lysine histone methyltransferase (HMTase) (EC 2.1.1.357) that specifically dimethylates nucleosomal histone H3 at lysine 36 and is involved in chromatin binding and in gene transcription regulation during various biological processes.

An exemplary amino acid sequence of human NSD2 polypeptide, found under UniProt ID O96028, is as follows:

| (SEQ ID NO: 1) | |
|---|---|
| MEFSIKQSPL SVQSVVKCIK MKQAPEILGS ANGKTPSCEV NRECSVFLSK AQLSSSLQEG | 60 |
| VMQKFNGHDA LPFIPADKLK DLTSRVFNGE PGAHDAKLRF ESQEMKGIGT PPNTTPIKNG | 120 |
| SPEIKLKITK TYMNGKPLFE SSICGDSAAD VSQSEENGQK PENKARRNRK RSIKYDSLLE | 180 |
| QGLVEAALVS KISSPSDKKI PAKKESCPNT GRDKDHLLKY NVGDLVWSKV SGYPWWPCMV | 240 |
| SADPLLHSYT KLKGQKKSAR QYHVQFFGDA PERAWIFEKS LVAFEGEGQF EKLCQESAKQ | 300 |
| APTKAEKIKL LKPISGKLRA QWEMGIVQAE EAASMSVEER KAKFTFLYVG DQLHLNPQVA | 360 |
| KEAGIAAESL GEMAESSGVS EEAAENPKSV REECIPMKRR RRAKLCSSAE TLESHPDIGK | 420 |
| STPQKTAEAD PRRGVGSPPG RKKTTVSMPR SRKGDAASQF LVFCQKHRDE VVAEHPDASG | 480 |
| EEIEELLRSQ WSLLSEKQRA RYNTKFALVA PVQAEEDSGN VNGKKRNHTK RIQDPTEDAE | 540 |
| AEDTPRKRLR TDKHSLRKRD TITDKTARTS SYKAMEAASS LKSQAATKNL SDACKPLKKR | 600 |
| NRASTAASSA LGFSKSSSPS ASLTENEVSD SPGDEPSESP YESADETQTE VSVSSKKSER | 660 |
| GVTAKKEYVC QLCEKPGSLL LCEGPCCGAF HLACLGLSRR PEGRFTCSEC ASGIHSCFVC | 720 |
| KESKTDVKRC VVTQCGKFYH EACVKKYPLT VFESRGFRCP LHSCVSCHAS NPSNPRPSKG | 780 |
| KMMRCVRCPV AYHSGDACLA AGCSVIASNS IICTAHFTAR KGKRHHAHVN VSWCFVCSKG | 840 |
| GSLLCCESCP AAFHPDCLNI EMPDGSWFCN DCRAGKKLHF QDIIWVKLGN YRWWPAEVCH | 900 |
| PKNVPPNIQK MKHEIGEFPV FFFGSKDYYW THQARVFPYM EGDRGSRYQG VRGIGRVFKN | 960 |
| ALQEAEARFR EIKLQREARE TQESERKPPP YKHIKVNKPY GKVQIYTADI SEIPKCNCKP | 1020 |
| TDENPCGFDS ECLNRMLMFE CHPQVCPAGE FCQNQCFTKR QYPETKIIKT DGKGWGLVAK | 1080 |
| RDIRKGEFVN EYVGELIDEE ECMARIKHAH ENDITHFYML TIDKDRIIDA GPKGNYSRFM | 1140 |
| NHSCQPNCET LKWTVNGDTR VGLFAVCDIP AGTELTFNYN LDCLGNEKTV CRCGASNCSG | 1200 |
| FLGDRPKTST TLSSEEKGKK TKKKTRRRRA KGEGKRQSED ECFRCGDGGQ LVLCDRKFCT | 1260 |
| KAYHLSCLGL GKRPFGKWEC PWHHCDVCGK PSTSFCHLCP NSFCKEHQDG TAFSCTPDGR | 1320 |
| SYCCEHDLGA ASVRSTKTEK PPPEPGKPKG KRRRRRGWRR VTEGK. | 1365 |

An exemplary human NSD2 gene sequence is available at Ensembl under Accession No. ENSG00000109685.19. The NSD2 gene is located on chromosome 4 at nucleotides 1,871,393-1,982,207 of the forward strand.

As used herein and unless otherwise indicated, the term “express” and “expression” refer to gene expression, include expression of nucleic acids (e.g., mRNA) and expression of polypeptides. Thus, “NSD2 expression” can be determined by evaluating expression of NSD2 mRNA and/or expression of NSD2 protein.

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” or “therapeutically effective amount” in connection with a compound means an amount capable of treating, preventing, or managing a disorder, disease or condition, or symptoms thereof.

As used herein and unless otherwise indicated, the term “subject” or “patient” includes an animal, and in some embodiments, a mammal. In some embodiments, a subject or patient is a human.

As used herein and unless otherwise indicated, the term “sample” refers to a sample obtained from a subject. The sample may be from any biological tissue or fluid. In some embodiments, a sample is derived from a human, e.g., a subject or a patient, e.g., a cancer patient, e.g., a multiple myeloma patient. A sample may include tissues, sections of tissues, cells, fluids, or extracts thereof, and can be isolated by any means, e.g., from blood, serum, biopsy, lymph node biopsy, bone marrow biopsy, needle biopsy, aspiration, etc.).

As used herein and unless otherwise indicated, the term “relapsed” refers to a disorder, disease, or condition that responded to treatment (e.g., achieved a partial or complete response) then had progression. The treatment can include one or more lines of therapy. In some embodiments, the disorder, disease or condition has been previously treated with one or more lines of therapy. In another embodiment, the disorder, disease or condition has been previously treated with one, two, three or four lines of therapy. In some embodiments, the disorder, disease or condition is a hematological malignancy.

As used herein and unless otherwise indicated, the term “refractory” refers to a disorder, disease, or condition that has not responded to prior treatment. In some embodiments, the disorder, disease, or condition has been previously treated one, two, three or four lines of therapy. In some embodiments, the disorder, disease, or condition has been previously treated with two or more lines of treatment, and did not respond to the most recent treatment. In some embodiments, the disorder, disease or condition is a hematological malignancy, and in particular, multiple myeloma.

In the context of a cancer, for example, a hematological malignancy, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In some embodiments, PFS means the time from treatment onset until tumor progression or death. In some embodiments, PFS means the time from the first dose of compound to the first occurrence of disease progression or death from any cause. In some embodiments, PFS rates are computed using the Kaplan-Meier estimates. Event-free survival (EFS) means the time from treatment onset until any treatment failure, including disease progression, treatment discontinuation for any reason, or death. In some embodiments, overall response rate (ORR) means the percentage of patients who achieve a response. In some embodiments, ORR means the sum of the percentage of patients who achieve complete and partial responses. In some embodiments, ORR means the percentage of patients whose best response≥partial response (PR). In some embodiments, duration of response (DoR) is the time from achieving a response until relapse or disease progression. In some embodiments, DoR is the time from achieving a response≥partial response (PR) until relapse or disease progression. In some embodiments, DoR is the time from the first documentation of a response until to the first documentation of progressive disease or death. In some embodiments, DoR is the time from the first documentation of a response≥partial response (PR) until to the first documentation of progressive disease or death. In some embodiments, time to response (TTR) means the time from the first dose of compound to the first documentation of a response. In some embodiments, TTR means the time from the first dose of compound to the first documentation of a response≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

As used herein “multiple myeloma” refers to hematological conditions characterized by malignant plasma cells and includes the following disorders: monoclonal gammopathy of undetermined significance (MGUS); low risk, intermediate risk, and high risk multiple myeloma; newly diagnosed multiple myeloma (including low risk, intermediate risk, and high risk newly diagnosed multiple myeloma); transplant eligible and transplant ineligible multiple myeloma; smoldering (indolent) multiple myeloma (including low risk, intermediate risk, and high risk smouldering multiple myeloma); active multiple myeloma; solitary plasmacytoma; extramedullary plasmacytoma; plasma cell leukemia; central nervous system multiple myeloma; light chain myeloma; non-secretory myeloma; Immunoglobulin D myeloma; and Immunoglobulin E myeloma; and multiple myeloma characterized by genetic abnormalities, such as Cyclin D translocations (for example, t(11;14)(q13;q32); t(6;14)(p21;32); t(12;14)(p13;q32); or t(6;20);); MMSET translocations (for example, t(4;14)(p16;q32)); MAF translocations (for example, t(14;16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)). In some embodiments, the multiple myeloma is characterized by a chromosomal translocation t(4;14). In some embodiments, the multiple myeloma is characterized according to the multiple myeloma International Staging System (ISS). In some embodiments, the multiple myeloma is Stage I multiple myeloma as characterized by ISS (e.g., serum β2 microglobulin<3.5 mg/L and serum albumin≥3.5 g/dL). In some embodiments, the multiple myeloma is Stage III multiple myeloma as characterized by ISS (e.g., serum β2 microglobulin>5.4 mg/L). In some embodiments, the multiple myeloma is Stage II multiple myeloma as characterized by ISS (e.g., not Stage I or III).

In certain embodiments, the treatment of multiple myeloma may be assessed by the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1-7), using the response and endpoint definitions shown below:

| Response | |
|---|---|
| Subcategory | Response Criteria^{a} |
| sCR | CR as defined below plus |
| | Normal FLC ratio and |
| | Absence of clonal cells in bone marrow^{b} by |
| | immunohistochemistry or immunofluorescence^{c} |
| CR | Negative immunofixation on the serum and urine and |
| | Disappearance of any soft tissue plasmacytomas |
| | and <5% plasma cells in bone marrow^{b} |
| VGPR | Serum and urine M-protein detectable by immunofixation but |
| | not on electrophoresis or 90% or greater reduction in serum |
| | M-protein plus urine M-protein level <100 mg per 24 h |
| PR | ≥50% reduction of serum M-protein and reduction in 24-h |
| | urinary M-protein by ≥90% or to <200 mg per 24 h |
| | If the serum and urine M-protein are unmeasurable,^{d} a ≥50% |
| | decrease in the difference between involved and uninvolved |
| | FLC levels is required in place of the M-protein criteria |
| | If serum and urine M-protein are unmeasurable, and serum free |
| | light assay is also unmeasurable, ≥50% reduction in plasma |
| | cells is required in place of M-protein, provided baseline bone |
| | marrow plasma cell percentage was ≥30% |
| | In addition to the above listed criteria, if present at baseline, |
| | a ≥50% reduction in the size of soft tissue plasmacytomas is |
| | also required |
| SD (not | Not meeting criteria for CR, VGPR, PR or progressive disease |
| recommended for | |
| use as an indicator | |
| of response; stability | |
| of disease is best | |
| described by | |
| providing the time | |
| to progression | |
| estimates) | |

Abbreviations: CR, complete response; FLC, free light chain; PR, partial response; SD, stable disease; sCR, stringent complete response; VGPR, very good partial response.
^{a}All response categories require two consecutive assessments made at any time before the institution of any new therapy; all categories also require no known evidence of progressive or new bone lesions if radiographic studies were performed. Radiographic studies are not required to satisfy these response requirements.
^{b}Confirmation with repeat bone marrow biopsy not needed.
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements: Bone marrow plasma cells ≥30%; Serum M-protein ≥1 g/dl (≥10 gm/l)[10 g/l]; Urine M-protein ≥200 mg/24 h; Serum FLC assay: Involved FLC level ≥10 mg/dl (≥100 mg/l); provided serum FLC ratio is abnormal.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below:

| Score | Description |
|---|---|
| 0 | Fully active, able to carry on all pre-disease |
| | performance without restriction |
| 1 | Restricted in physically strenuous activity but |
| | ambulatory and able to carry out work of a light |
| | or sedentary nature, eg, light housework, office work. |
| 2 | Ambulatory and capable of all self-care but unable |
| | to carry out any work activities. Up and about more |
| | than 50% of waking hours. |
| 3 | Capable of only limited self-care, confined to bed or |
| | chair more than 50% of waking hours. |
| 4 | Completely disabled. Cannot carry on any self-care. |
| | Totally confined to bed or chair |
| 5 | Dead |

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography) scan, MRI (magnetic resonance imaging) of the brain and spine, CSF (cerebrospinal fluid), ophthalmologic exams, vitreal fluid sampling, retinal photograph, bone marrow evaluation and other commonly accepted evaluation modalities.

As used herein and unless otherwise indicated, the terms “co-administration” and “in combination with” include the administration of one or more therapeutic agents (for example, a compound provided herein and another anti-cancer agent or supportive care agent) simultaneously, concurrently, or sequentially with no specific time limits. In some embodiments, the agents are present in the cell or in the patient's body at the same time or exert their biological or therapeutic effect at the same time. In some embodiments, the therapeutic agents are in the same composition or unit dosage form. In other embodiments, the therapeutic agents are in separate compositions or unit dosage forms.

The term “supportive care agent” refers to any substance that treats, prevents or manages an adverse effect from treatment with another therapeutic agent.

As used herein, “induction therapy” refers to the first treatment given for a disease, or the first treatment given with the intent of inducing complete remission in a disease, such as cancer. When used by itself, induction therapy is the one accepted as the best available treatment. If residual cancer is detected, patients are treated with another therapy, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

As used herein “transplant” refers to high-dose therapy with stem cell rescue. Hematopoietic (blood) or bone marrow stem cells are used not as treatment but to rescue the patient after the high-dose therapy, for example high dose chemotherapy and/or radiation. Transplant includes “autologous” stem cell transplant (ASCT), which refers to use of the patients' own stem cells being harvested and used as the replacement cells. In some embodiments, transplant also includes tandem transplant or multiple transplants.

The term “biological therapy” refers to administration of biological therapeutics such as cord blood, stem cells, growth factors and the like.

### B. Selection of Subjects for Treatment

In some embodiments, provided herein are methods of treating multiple myeloma, comprising administering to a subject with multiple myeloma an inhibitor of Nuclear Receptor Binding SET Domain Protein 2 (NSD2).

In some embodiments, provided herein are methods of selecting a subject with multiple myeloma for treatment with an NSD2 inhibitor, comprising determining whether the subject has a 4:14 chromosome translocation (t(4;14)). In some embodiments, if the subject has a t(4;14), the subject is selected for treatment with an NSD2 inhibitor.

In some embodiments, provided herein are methods of predicting whether a subject with multiple myeloma will benefit from treatment with an NSD2 inhibitor, comprising determining whether the subject has a 4:14 chromosome translocation (t(4;14)). In some embodiments, if the subject has a t(4;14) translocation, the subject is predicted to benefit from treatment with an NSD2 inhibitor.

The subject may be identified as having multiple myeloma by any available method. In some embodiments, the subject has previously been determined to have multiple myeloma.

In some embodiments, cells of the cancer have a chromosomal rearrangement that produces a t(4;14) chromosomal translocation. In some embodiments, the t(4;14) chromosomal translocation is in the NSD2 gene.

In some embodiments, cells of the cancer have previously been determined to have a t(4;14) chromosomal translocation.

In some embodiments, the t(4;14) chromosomal translocation results in a disruption in the NSD2 gene.

In some embodiments, the t(4;14) chromosomal translocation does not result in a disruption of the protein coding region of the NSD2 gene.

In some embodiments, the t(4;14) chromosomal translocation results in a disruption of the protein coding region of the NSD2 gene. In some embodiments, translocations that disrupt the protein coding region of the NSD2 gene correlate with shorter overall survival.

In some embodiments, the multiple myeloma expresses a truncated NSD2 protein. In some embodiments, the multiple myeloma expresses a fusion protein that comprises a truncated or full-length NSD2 protein. In some embodiments, patients expressing truncated fusion proteins comprising NSD2 have a worse overall survival than patients expressing the full coding fusion transcript or no fusion transcript.

In some embodiments, the disruption in the NSD2 gene is located in the first coding exon of the NSD2 gene.

In some embodiments, the disruption in the NSD2 gene is an early disruption, based on the position of the translocation breakpoint. An early disruption is a disruption between the transcription start site of the NSD2 gene and the translation start site of the NSD2 gene. In some embodiments, an early disruption is between genomic position 1,871,393 and genomic position 1,900,655 of GRCh38.p13.

In some embodiments, the disruption in the NSD2 gene is a late disruption, based on the position of the translocation breakpoint. In some embodiments, a late disruption is a disruption in the NSD2 gene after the translation start site. In some embodiments, a late disruption is a disruption downstream of genomic position 1,900,655 of GRCh38.p13. In some embodiments, a late disruption is a disruption between genomic position 1,900,655 and genomic position 1,983,934 of GRCh38.p13.

In some embodiments, the 4:14 chromosome translocation is located before genomic position 1,871,393 of Genome Reference Consortium Human Build 38 patch release 13 (“GRCh38.p13”). In some such embodiments, there is no disruption in the NSD2 gene.

In some embodiments, the t(4;14) is located after the transcription start site of the NSD2 gene. In some embodiments, the t(4;14) is located between the start of the first protein-coding exon and the start of the second protein-coding exon of the NSD2 gene. In some embodiments, the t(4;14) is located at or after the start of the second protein-coding exon of the NSD2 gene.

In some embodiments, the t(4;14) is located at or after genomic position 1,871,393 of GRCh38.p13. In some embodiments, the t(4;14) is located between genomic position 1,871,393 and genomic position 1,900,655 of GRCh38.p13.

In some embodiments, the t(4;14) is located at or after genomic position 1,900,655 of GRCh38.p13. In some embodiments, the t(4;14) is located between genomic position 1,900,655 and genomic position 1,983,934 of GRCh38.p13

In some embodiments, the t(4;14) chromosomal translocation is identified, directly or indirectly, by analyzing NSD2 protein expression. In some embodiments, NSD2 protein expression is determined and compared to a reference (e.g., a reference sample, or a reference value, or any other comparison to which is indicative of whether, or to what extent, and/or in what form, the cancer expresses the NSD2 polypeptide). In some embodiments, NSD2 expression in a multiple myeloma is determined, relative to NSD2 expression in a non-cancerous cell. In some embodiments, analyzing and quantifying NSD2 expression in patient samples, cells, and/or cell lines is determined by immunohistochemical and/or immunofluorescence techniques.

In some embodiments, the t(4;14) chromosomal translocation is identified by a method comprising polymerase chain reaction (PCR), reverse-transcription-PCR (RT-PCR, including real-time RT-PCR, qRT-PCR), RNA sequencing (RNA-seq), in situ hybridization (ISH), fluorescence in situ hybridization (FISH), transcript in situ hybridization, whole genome sequencing (WGS), whole exome sequencing (WES), multiplex ligation-dependent probe assays (MLPA), mass spectrometry (MS), and/or matrix assisted laser desorption ionization-time of flight MS (MALDI-TOF MS).

The term “polymerase chain reaction,” or “PCR,” as used herein refers to a procedure wherein small amounts of a nucleic acid, RNA and/or DNA, are amplified. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc.

Other PCR-based methods can also be used. Examples of PCR methods can be found in the literature. Examples of PCR assays can be found, for example, in U.S. Pat. No. 6,927,024. Nonlimiting examples of RT-PCR methods can be found in U.S. Pat. No. 7,122,799. A nonlimiting method of fluorescent in situ PCR is described in U.S. Pat. No. 7,186,507.

In some embodiments, Real-Time Reverse Transcription-PCR (qRT-PCR) can be used for both the detection and quantification of RNA targets (Bustin, et al., 2005, Clin. Sci., 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

The term “RNA sequencing” or “RNA-Seq” refers to a method typically including (1) isolating RNA; (2) depleting ribosomal RNA; (3) cDNA synthesis; and (4) sequencing cDNA by next-generation sequencing (NGS). Techniques for RNA-Seq are well known to one skilled in the art and methods are described in Waern et al., Methods Mol Biol., 2011, 759: 125-132; Wilhelm et al., Nature Protocols, 2010, 5(2):255-66; and Hoeijmakers et al., Methods Mol Biol., 2013, 923:221-39.

The term “in situ hybridization” or “ISH” refers to techniques that can be used to determine mRNA levels (reviewed by A. K. Raap (1998) Mutat. Res. 400:287-298). In situ hybridization techniques allow the visual detection of mRNA in a cell by incubating the cell with a labeled (e.g., fluorescently labeled or digoxigenin labeled) oligonucleotide probe that hybridizes to the mRNA of interest, and then examining the cell by microscopy.

For more accurate gene mapping, fluorescence in situ hybridization (FISH) techniques can be used. In particular, high-resolution FISH techniques (A. Palotie et al. (1996) Ann. Med. 28:101-106) utilize free chromatin, DNA fibers, or mechanically-stretched chromosomes to map gene sequences ranging from several kilobases to 300-kb in size. Alternatively, the chromosomal location of a gene can be determined from the appropriate genome database, for example, the Homo sapiens genome database available at the Entrez Genome website (National Center for Biotechnology Information, Bethesda, Md.).

The term “whole genome sequencing” or “WGS,” as used herein, refers to a method of sequencing the entire genome.

The term “whole exome sequencing” or “WES,” as used herein, refers to a method of sequencing all the protein-coding regions (exons) of the genome.

The term “mass spectrometry” or “mass spec” or “MS” as used herein, refers to an analytical technique for measuring the mass-to-charge ratio of ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer. Exemplary mass spectrometry analytical techniques include electrospray ionization mass spectroscopy (ESI), high resolution mass spectrometry (HRMS), liquid chromatography mass spectrometry (LCMS), and liquid chromatography-tandem mass spectrometry (LC-MS/MS). Also exemplary is MALDI mass spectrometry, such as MALDI-TOF mass spectrometry, wherein matrix-assisted laser desorption/ionization (MALDI) is the ion source, and the mass analyzer is time-of-flight (TOF) mass spectrometer.

### C. Inhibitors of Nuclear Receptor Binding SET Domain Protein 2 (NSD2)

In some embodiments, an inhibitor of NSD2 is an antibody. The term “antibody” is used herein in the broadest sense and covers fully assembled antibodies, antibody fragments that retain the ability to specifically bind to the antigen (e.g., Fab, F(ab′)₂, Fv, and other fragments), single chain antibodies, diabodies, antibody chimeras, hybrid antibodies, bispecific antibodies, humanized antibodies, and the like.

In some embodiments, an inhibitor of NSD2 is a small molecule. The term “small molecule” is used herein in the broadest sense and covers a molecule of less than 1,000 daltons, such as synthetic inorganic, organometallic, and organic molecule.

In some embodiments, an inhibitor of NSD2 is an aptamer. The term “aptamer” as used herein is an oligonucleotide or a peptide molecule that specifically binds to a target. In some embodiments, the aptamer is an oligonucleotide having, e.g., about 15 to about 100 nucleotides, such as about 15 to about 50 nucleotides.

In some embodiments, an inhibitor of NSD2 is a small interfering RNA (siRNA). The term “siRNA” or “small interfering RNA” or “short interfering RNA” or “silencing RNA” as used herein refers to a single- or double-stranded non-coding RNA, from about 20 to about 27 bases or base pairs in length, which interferes with expression of specific RNAs having complementary nucleotide sequences. In some embodiments an siRNA causes degradation of a mRNA, preventing translation of the protein product.

In some embodiments, an inhibitor of NSD2 is an antisense oligonucleotide. The term “antisense oligonucleotide” as used herein refers to a single stranded oligonucleotide that is complementary to a particular sequence in a target gene or RNA, and modulates its expression or splicing. In some embodiments, the antisense oligonucleotide is at least 10, such as at least 15 nucleotides, and optionally between about 15 to about 30, such as about 15 to about 25 nucleotides, and may contain one or more modifications compared to naturally-occurring nucleotides.

In some embodiments, the specific amount of the inhibitor of NSD2 provided herein for use in the methods provided herein is determined by factors such as the specific type of inhibitor used, the type of multiple myeloma being treated or managed, the severity and stage of disease, the age, height, and/or weight of the subject being treated; and any optional additional active agents concurrently administered to the patient.

### D. Methods of Use

In some embodiments, provided herein is a method of treating multiple myeloma, comprising administering to a subject with multiple myeloma an inhibitor of Nuclear Receptor Binding SET Domain Protein 2 (NSD2).

In some embodiments, provided herein is a method of treating multiple myeloma, comprising administering to a subject with multiple myeloma a therapeutically effective amount of an inhibitor of Nuclear Receptor Binding SET Domain Protein 2 (NSD2).

In some embodiments, the multiple myeloma is plasma cell leukemia (PCL).

In some embodiments, the multiple myeloma is newly diagnosed multiple myeloma.

In some embodiments, the multiple myeloma is relapsed or refractory. In some embodiments, the multiple myeloma is refractory to lenalidomide. In some embodiment, the multiple myeloma is refractory to pomalidomide. In some embodiments, the multiple myeloma is refractory to the combination of pomalidomide and a proteasome inhibitor. In some embodiments, the proteasome inhibitor is selected from bortezomib, carfilzomib, and ixazomib. In some embodiments, the multiple myeloma is refractory to the combination of pomalidomide and an inflammatory steroid. In some embodiments, the inflammatory steroid is selected from dexamethasone or prednisone. In some embodiments, the multiple myeloma is refractory to the combination of pomalidomide and a CD38 directed monoclonal antibody.

In some embodiment, provided herein are methods for achieving a complete response, partial response, or stable disease in a patient, comprising administering to subject with multiple myeloma an inhibitor of NSD2.

In some embodiments, also provided herein are methods for inducing a therapeutic response assessed with the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1 7) of a patient, comprising administering to a subject with multiple myeloma an inhibitor of NSD2.

In some embodiments, provided herein are methods for achieving a stringent complete response, complete response, or very good partial response, as determined by the International Uniform Response Criteria for Multiple Myeloma (IURC) in a patient, comprising administering to a subject with multiple myeloma an inhibitor of NSD2.

In some embodiments, provided herein are methods for achieving an increase in overall survival, progression-free survival, event-free survival, time to progression, or disease-free survival in a patient, comprising administering to a subject with multiple myeloma an inhibitor of NSD2.

Also provided herein, in some embodiments, is a method of selecting a subject with multiple myeloma for treatment with an NSD2 inhibitor, comprising determining whether the subject has a 4:14 chromosome translocation (t(4;14)), wherein if the subject has a t(4;14), the subject is selected for treatment with an NSD2 inhibitor.

In some embodiments, provided herein is a method of selecting a subject with multiple myeloma for treatment with an NSD2 inhibitor, comprising:
a) obtaining a sample from the subject;
b) determining whether the subject has a 4:14 chromosome translocation (t(4;14));
c) if the subject has a t(4;14) chromosomal translocation, selecting the subject for treatment with an NSD2 inhibitor.

In some embodiments, provided herein is a method of predicting whether a subject with multiple myeloma will benefit from treatment with an NSD2 inhibitor, comprising determining whether the subject has a 4:14 chromosome translocation (t(4;14)), wherein if the subject has a t(4;14) translocation, the subject is predicted to benefit from treatment with an NSD2 inhibitor.

In some embodiments, provided herein is a method of predicting whether a subject with multiple myeloma will benefit from treatment with an NSD2 inhibitor, comprising:
a) obtaining a sample from the subject;
b) determining whether the subject has a 4:14 chromosome translocation (t(4;14));
c) if the subject has a t(4;14) translocation, the subject is predicted to benefit from treatment with an NSD2 inhibitor.

Also provided herein are methods of treating patients who have been previously treated for multiple myeloma but are non-responsive to standard therapies, as well as those who have not previously been treated. Further encompassed are methods of treating patients who have undergone surgery in an attempt to treat multiple myeloma, as well as those who have not. Also provided herein are methods of treating patients who have been previously undergone transplant therapy, as well as those who have not.

The methods provided herein include treatment of multiple myeloma that is relapsed, refractory or resistant. The methods provided herein include prevention of multiple myeloma that is relapsed, refractory or resistant. The methods provided herein include management of multiple myeloma that is relapsed, refractory or resistant. In some such embodiments, the myeloma is primary, secondary, tertiary, quadruply, or quintuply relapsed multiple myeloma. In some embodiments, the methods provided herein reduce, maintain or eliminate minimal residual disease (MRD). In some embodiments, provided herein is a method of increasing rate and/or durability of MRD negativity in multiple myeloma patients, comprising administering to a subject with multiple myeloma an inhibitor of NSD2. In some embodiments, methods provided herein encompass treating, preventing or managing various types of multiple myeloma, such as monoclonal gammopathy of undetermined significance (MGUS), low risk, intermediate risk, and high risk multiple myeloma, newly diagnosed multiple myeloma (including low risk, intermediate risk, and high risk newly diagnosed multiple myeloma), transplant eligible and transplant ineligible multiple myeloma, smoldering (indolent) multiple myeloma (including low risk, intermediate risk, and high risk smouldering multiple myeloma), active multiple myeloma, solitary plasmacytoma, extramedullary plasmacytoma, plasma cell leukemia, central nervous system multiple myeloma, light chain myeloma, non-secretory myeloma, Immunoglobulin D myeloma, and Immunoglobulin E myeloma, by administering to a subject with multiple myeloma an inhibitor of NSD2.

In another embodiment, methods provided herein encompass treating, preventing or managing multiple myeloma characterized by genetic abnormalities other than, or in addition to, t(4;14), such as Cyclin D translocations (for example, t(11;14)(q13;q32); t(6;14)(p21;32); t(12;14)(p13;q32); or t(6;20);); MMSET translocations (for example, t(4;14)(p16;q32)); MAF translocations (for example, t(14;16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)), by administering an inhibitor of NSD2. In some embodiments, the multiple myeloma is characterized according to the multiple myeloma International Staging System (ISS). In some embodiments, the multiple myeloma is Stage I multiple myeloma as characterized by ISS (e.g., serum β2 microglobulin<3.5 mg/L and serum albumin≥3.5 g/dL). In some embodiments, the multiple myeloma is Stage III multiple myeloma as characterized by ISS (e.g., serum β2 microglobulin>5.4 mg/L). In one embodiment, the multiple myeloma is Stage II multiple myeloma as characterized by ISS (e.g., not Stage I or III).

In some embodiments, the methods comprise administering an inhibitor of NSD2. In some embodiments, the methods comprise administering an inhibitor of NSD2 as consolidation therapy. In some embodiments, the methods comprise administering an inhibitor of NSD2 as maintenance therapy.

In one particular embodiment of the methods described herein, the multiple myeloma is plasma cell leukemia.

In some embodiments, the multiple myeloma is high risk multiple myeloma. In some such embodiments, the high risk multiple myeloma is relapsed or refractory. In some embodiments, the high risk multiple myeloma is multiple myeloma that is relapsed within 12 months of first treatment. In some embodiments, the high risk multiple myeloma is multiple myeloma that is additionally characterized by genetic abnormalities, for example, one or more of del(17/17p) and t(14;16)(q32;q32). In some such embodiments, the high risk multiple myeloma is relapsed or refractory to one, two or three previous treatments.

In some embodiments, the multiple myeloma is additionally characterized by a p53 mutation. In some embodiments, the p53 mutation is a Q331 mutation. In some embodiments, the p53 mutation is an R273H mutation. In some embodiments, the p53 mutation is a K132 mutation. In some embodiments, the p53 mutation is a K132N mutation. In some embodiments, the p53 mutation is an R337 mutation. In some embodiments, the p53 mutation is an R337L mutation. In some embodiments, the p53 mutation is a W146 mutation. In some embodiments, the p53 mutation is an S261 mutation. In some embodiments, the p53 mutation is an S261T mutation. In some embodiments, the p53 mutation is an E286 mutation. In some embodiments, the p53 mutation is an E286K mutation. In some embodiments, the p53 mutation is an R175 mutation. In some embodiments, the p53 mutation is an R175H mutation. In some embodiments, the p53 mutation is an E258 mutation. In some embodiments, the p53 mutation is an E258K mutation. In some embodiments, the p53 mutation is an A161 mutation. In some embodiments, the p53 mutation is an A161T mutation.

In some embodiments, the multiple myeloma is characterized by homozygous deletion of p53. In some embodiments, the multiple myeloma is characterized by homozygous deletion of wild type p53.

In some embodiments, the multiple myeloma is characterized by wild type p53.

In some embodiments, the multiple myeloma is characterized by activation of one or more oncogenic drivers. In some embodiments, the one or more oncogenic drivers are selected from the group consisting of C-MAF, MAFB, FGFR3, MMset, Cyclin D1, and Cyclin D. In some embodiments, the multiple myeloma is characterized by activation of C MAF. In some embodiments, the multiple myeloma is characterized by activation of MAFB. In some embodiments, the multiple myeloma is characterized by activation of FGFR3 and MMset. In some embodiments, the multiple myeloma is characterized by activation of C MAF, FGFR3, and MMset. In certain embodiments, the multiple myeloma is characterized by activation of Cyclin D1. In some embodiments, the multiple myeloma is characterized by activation of MAFB and Cyclin D1. In some embodiments, the multiple myeloma is characterized by activation of Cyclin D.

In some embodiments, the multiple myeloma is characterized by one or more chromosomal translocations other than, or in addition to, the chromosomal translocation t(4;14). In some embodiments, the chromosomal translocations are t(4;14) and t(14;16). In some embodiments, the chromosomal translocation translocations are t(4;14) and t(14;20). In some embodiments, the chromosomal translocations are t(4;14) and t(11;14). In some embodiments, the chromosomal translocations are t(4;14) and t(6;20). In some embodiments, the chromosomal translocations are t(4;14) and t(20;22). In some embodiments, the chromosomal translocations are t(4;14) and t(6;20) and t(20;22). In some embodiments, the chromosomal translocations are t(4;14) and t(16;22). In some embodiments, the chromosomal translocations are t(4;14) and t(14;16) and t(16;22). In some embodiments, the chromosomal translocations are t(4;14) and t(14;20) and t(11;14).

In some embodiments, the multiple myeloma is characterized by a Q331 p53 mutation, by activation of C-MAF, and by chromosomal translocations at t(4;14) and at t(14;16). In some embodiments, the multiple myeloma is characterized by homozygous deletion of p53, by activation of C-MAF, and by chromosomal translocations at t(4;14) and at t(14;16). In some embodiments, the multiple myeloma is characterized by a K132N p53 mutation, by activation of MAFB, and by chromosomal translocations at t(4;14) and at t(14;20). In some embodiments, the multiple myeloma is characterized by wild type p53, by activation of FGFR3 and MMset, and by a chromosomal translocation at t(4;14). In some embodiments, the multiple myeloma is characterized by wild type p53, by activation of C-MAF, and by chromosomal translocations at t(4;14) and at t(14;16). In some embodiments, the multiple myeloma is characterized by homozygous deletion of p53, by activation of FGFR3, MMset, and C MAF, and by chromosomal translocations at t(4;14) and t(14;16). In some embodiments, the multiple myeloma is characterized by homozygous deletion of p53, by activation of Cyclin D1, and by chromosomal translocations at t(4;14) and at t(11;14). In some embodiments, the multiple myeloma is characterized by an R337L p53 mutation, by activation of Cyclin D1, and by chromosomal translocations at t(4;14) and at t(11;14). In some embodiments, the multiple myeloma is characterized by a W146 p53 mutation, by activation of FGFR3 and MMset, and by chromosomal translocations at t(4;14) and at t(4;14). In some embodiments, the multiple myeloma is characterized by an S261T p53 mutation, by activation of MAFB, and by chromosomal translocations at t(4;14) and at t(6;20) and t(20;22). In some embodiments, the multiple myeloma is characterized by an E286K p53 mutation, by activation of FGFR3 and MMset, and by a chromosomal translocation at t(4;14). In some embodiments, the multiple myeloma is characterized by an R175H p53 mutation, by activation of FGFR3 and MMset, and by a chromosomal translocation at t(4;14). In some embodiments, the multiple myeloma is characterized by an E258K p53 mutation, by activation of C-MAF, and by chromosomal translocations at t(4;14) and at t(14;16) and t(16;22). In some embodiments, the multiple myeloma is characterized by wild type p53, by activation of MAFB and Cyclin D1, and by chromosomal translocations at t(4;14) and at t(14;20) and t(11;14). In some embodiments, the multiple myeloma is characterized by an A161T p53 mutation, by activation of Cyclin D, and by chromosomal translocations at t(4;14) and at t(11;14).

In some embodiments of the methods described herein, the multiple myeloma is transplant eligible newly diagnosed multiple myeloma. In another embodiment, the multiple myeloma is transplant ineligible newly diagnosed multiple myeloma.

In yet other embodiments, the multiple myeloma is characterized by early progression (for example less than 12 months) following initial treatment. In still other embodiments, the multiple myeloma is characterized by early progression (for example less than 12 months) following autologous stem cell transplant. In some embodiments, the multiple myeloma is refractory to lenalidomide. In some embodiments, the multiple myeloma is refractory to pomalidomide. In some such embodiments, the multiple myeloma is predicted to be refractory to pomalidomide (for example, by molecular characterization). In some embodiments, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In certain embodiments, provided herein are methods of treating, preventing, and/or managing multiple myeloma, including relapsed/refractory multiple myeloma in a subject with impaired renal function or a symptom thereof, comprising administering an inhibitor of NSD2, to a subject having relapsed/refractory multiple myeloma with impaired renal function.

In certain embodiments, provided herein are methods of treating, preventing, and/or managing multiple myeloma, including relapsed or refractory multiple myeloma in a frail subject, comprising administering an inhibitor of NSD2, to a frail subject having multiple myeloma. In some such embodiments, the frail subject is characterized by ineligibility for induction therapy, or intolerance to dexamethasone treatment. In some such embodiment the frail subject is elderly, for example, older than 65 years old.

In certain embodiments, provided herein are methods of treating, preventing or managing multiple myeloma, comprising administering to a subject an inhibitor of NSD2, wherein the multiple myeloma is fourth line relapsed/refractory multiple myeloma.

In certain embodiments, provided herein are methods of treating, preventing or managing multiple myeloma, comprising administering to a subject an inhibitor of NSD2, as induction therapy, wherein the multiple myeloma is newly diagnosed, transplant-eligible multiple myeloma.

In certain embodiments, provided herein are methods of treating, preventing or managing multiple myeloma, comprising administering to a subject an inhibitor of NSD2, as maintenance therapy after other therapy or transplant, wherein the multiple myeloma is newly diagnosed, transplant-eligible multiple myeloma prior to the other therapy or transplant.

In certain embodiments, provided herein are methods of treating, preventing or managing multiple myeloma, comprising administering to a subject an inhibitor of NSD2, as maintenance therapy after other therapy or transplant. In some embodiments, the multiple myeloma is newly diagnosed, transplant-eligible multiple myeloma prior to the other therapy and/or transplant. In some embodiments, the other therapy prior to transplant is treatment with chemotherapy or an inhibitor of NSD2.

In certain embodiments, provided herein are methods of treating, preventing or managing multiple myeloma, comprising administering to a subject an inhibitor of NSD2, wherein the multiple myeloma is high risk multiple myeloma, that is relapsed or refractory to one, two or three previous treatments.

In certain embodiments, provided herein are methods of treating, preventing or managing multiple myeloma, comprising administering to a subject an inhibitor of NSD2, wherein the multiple myeloma is newly diagnosed, transplant-ineligible multiple myeloma.

In certain embodiments, the subject to be treated with one of the methods provided herein has not been treated with multiple myeloma therapy prior to the administration of an inhibitor of NSD2. In certain embodiments, the subject to be treated with one of the methods provided herein has been treated with multiple myeloma therapy prior to the administration of an inhibitor of NSD2. In certain embodiments, the subject to be treated with one of the methods provided herein has developed drug resistance to the anti-multiple myeloma therapy. In some such embodiments, the subject has developed resistance to one, two, or three anti-multiple myeloma therapies, wherein the therapies are selected from a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide).

The methods provided herein encompass treating a subject regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old. In some embodiments, the subject is an elderly multiple myeloma subject, such as a subject older than 65 years old. In some embodiments, the subject is an elderly multiple myeloma subject, such as a subject older than 75 years old.

### E. Combination Therapy with at Least One Second Therapeutic Agent

In some embodiments, the methods provided herein (use of an inhibitor of NSD2) additionally comprises administering to the subject at least one second therapeutic agent, also referred to herein as an “additional agent” or “additional active agent.”

In some embodiments, the at least one second therapeutic agent is selected from a chemotherapy agent, a steroid, an immunomodulating agent, a proteasome inhibitor, a histone deacetylase inhibitor, an anti-CD38 antibody, and anti-SLAMF7 antibody, an antibody-drug conjugate, and a nuclear export inhibitor.

In some embodiments, the at least one second therapeutic agent is selected from lenalidomide, thalidomide, pomalidomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, selinexor, pamidronate, zoledronic acid, and denosumab.

In some embodiments, the at least one second therapeutic agent is lenalidomide. In some embodiments, the at least one second therapeutic agent is iberdomide. In some embodiments, the at least one second therapeutic agent is (S)-4-(4-(4-(((2-(2,6-dioxopiperidin-3-yl)-1-oxoisoindolin-4-yl)oxy)methyl)benzyl)piperazin-1-yl)-3-fluorobenzonitrile. In some embodiments, the at least one second therapeutic agent is (i) lenalidomide, pomalidomide, or thalidomide; and (ii) dexamethasone. In some embodiments, the at least one second therapeutic agent is (i) carfilzomib, ixazomib, or bortezomib; (ii) lenalidomide; and (iii) dexamethasone. In some embodiments, the at least one second therapeutic agent is (i) bortezomib or carfilzomib; (ii) cyclophosphamide; and (iii) dexamethasone. In some embodiments, the at least one second therapeutic agent is (i) elotuzumab or daratumumab; (ii) lenalidomide; and (iii) dexamethasone. In some embodiments, the at least one second therapeutic agent is bortezomib, liposomal doxorubicin, and dexamethasone. In some embodiments, the at least one second therapeutic agent is panobinostat, bortezomib, and dexamethasone. In some embodiments, the at least one second therapeutic agent is elotuzumab, bortezomib, and dexamethasone. In some embodiments, the at least one second therapeutic agent is melphalan and prednisone, with or without thalidomide or bortezomib. In some embodiments, the at least one second therapeutic agent is vincristine, doxorubicin, and dexamethasone. In some embodiments, the at least one second therapeutic agent is dexamethasone, cyclophosphamide, etoposide, and cisplatin. In some embodiments, the at least one second therapeutic agent is dexamethasone, thalidomide, cisplatin, doxorubicin, cyclophosphamide, and etoposide, with or without bortezomib.

In some embodiments, the at least one second therapeutic agent is a steroid.

In some embodiments, the specific amount (dosage) of the at least one second therapeutic agent provided herein as used in the methods provided herein is determined by factors such as the specific agent used, the type of multiple myeloma being treated or managed, the severity and stage of disease, the amount of an inhibitor of NSD2 provided herein, and any optional additional active agents concurrently administered to the patient.

In some embodiments, the dosage of an at least one second therapeutic agent provided herein as used in the methods provided herein is determined based on a commercial package insert of medicament (e.g., a label) as approved by the FDA or a similar regulatory agency of a country other than the USA for said active agent. In some embodiments, the dosage of a second therapeutic agent provided herein as used in the methods provided herein is a dosage approved by the FDA or a similar regulatory agency of a country other than the USA for said therapeutic agent. In some embodiments, the dosage of a second therapeutic agent provided herein as used in the methods provided herein is a dosage used in a human clinical trial for said therapeutic agent. In some embodiments, the dosage of a second therapeutic agent provided herein as used in the methods provided herein is lower than a dosage approved by the FDA or a similar regulatory agency of a country other than the USA for said therapeutic agent or a dosage used in a human clinical trial for said active agent, depending on, e.g., the synergistic effects between the second therapeutic agent and an inhibitor of NSD2 as provided herein.

The combined use of an inhibitor of NSD2 provided herein can also be combined or used in conjunction with (e.g. before, during, or after) conventional therapy including, but not limited to, surgery, biological therapy (including immunotherapy, for example with checkpoint inhibitors), radiation therapy, chemotherapy, stem cell transplantation, cell therapy, or other non-drug based therapy presently used to treat, prevent or manage cancer (e.g., multiple myeloma). The combined use of an inhibitor of NSD2 provided herein and conventional therapy may provide a unique treatment regimen that is unexpectedly effective in certain patients. Without being limited by theory, it is believed that an inhibitor of NSD2 provided herein may provide additive or synergistic effects when given concurrently with conventional therapy.

As discussed elsewhere herein, encompassed herein is a method of reducing, treating and/or preventing adverse or undesired effects associated with conventional therapy including, but not limited to, surgery, chemotherapy, radiation therapy, biological therapy and immunotherapy. An inhibitor of NSD2 provided herein, and an at least one second therapeutic agent ingredient can be administered to a patient prior to, during, or after the occurrence of the adverse effect associated with conventional therapy. In some embodiments, the at least one second therapeutic agent is dexamethasone.

The inhibitor of NSD2 provided herein can also be further combined or used in combination with other therapeutic agents useful in the treatment and/or prevention of multiple myeloma described herein. In one such embodiment, the at least one second therapeutic agent is dexamethasone.

In some embodiments, provided herein is a method of treating, preventing, or managing multiple myeloma, comprising administering to a patient an inhibitor of NSD2 provided herein, further in combination with one or more additional therapeutic agents, and optionally further in combination with radiation therapy, blood transfusions, or surgery.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such as an inhibitor of NSD2 provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., an at least one second therapeutic agent) to the subject. The first therapy and the second therapy independently can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a third therapy (e.g., an additional prophylactic or therapeutic agent) to the subject. Quadruple therapy is also contemplated herein, as is quintuple therapy. In some embodiments, the second therapy is dexamethasone.

Administration of an inhibitor of NSD2 provided herein, and one or more second therapeutic agents to a subject can occur simultaneously or sequentially by the same or different routes of administration. The suitability of a particular route of administration employed for a particular therapeutic agent will depend on the active agent itself (e.g., whether it can be administered orally without decomposing prior to entering the blood stream).

The route of administration of an inhibitor of NSD2 provided herein is independent of the additional therapy. In some embodiments, an inhibitor of NSD2 provided herein is administered orally. In another embodiment, an inhibitor of NSD2 provided herein is administered intravenously. Thus, in accordance with these embodiments, an inhibitor of NSD2 provided herein is administered orally or intravenously, and the additional therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In some embodiments, an inhibitor of NSD2 provided herein, and an additional therapy are administered by the same mode of administration, orally or by IV. In another embodiment, an inhibitor of NSD2 provided herein is administered by one mode of administration, e.g., by IV, whereas the additional agent (an anti-multiple myeloma agent) is administered by another mode of administration, e.g., orally.

In some embodiments, the at least one second therapeutic agent is administered intravenously or subcutaneously and once or twice daily in an amount of from about 1 to about 1000 mg, from about 5 to about 500 mg, from about 10 to about 350 mg, or from about 50 to about 200 mg. The specific amount of the additional active agent will depend on the specific agent used, the type of multiple myeloma being treated or managed, the severity and stage of disease, the amount of inhibitor of NSD2 provided herein, and any optional additional active agents concurrently administered to the subject.

One or more additional active ingredients or agents can be used together with an inhibitor of NSD2 provided herein in the methods and compositions provided herein. Additional active agents can be large molecules (e.g., proteins), small molecules (e.g., synthetic inorganic, organometallic, or organic molecules), or cell therapies (e.g., CAR cells).

Examples of additional active agents that can be used in the methods and compositions described herein include one or more of melphalan, vincristine, cyclophosphamide, etoposide, doxorubicin, bendamustine, obinutuzmab, a proteasome inhibitor (for example, bortezomib, carfilzomib, ixazomib, oprozomib or marizomib), a deacetylase inhibitor, such as a histone deacetylase inhibitor (as described herein, for example, panobinostat, ACY241), a BET inhibitor (for example, GSK525762A, OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one, EP11313 and EP11336), a BCL2 inhibitor (for example, venetoclax or navitoclax), an MCL-1 inhibitor (for example, AZD5991, AMG176, MIK665, S64315, or S63845), an LSD-1 inhibitor (for example, ORY-1001, ORY-2001, INCB-59872, IMG-7289, TAK-418, GSK-2879552, 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof), a corticosteroid (for example, prednisone), dexamethasone; an antibody (for example, a CS1 antibody, such as elotuzumab; a CD38 antibody, such as daratumumab or isatuximab; or a BCMA antibody or antibody-conjugate, such as GSK2857916 or BI 836909), a checkpoint inhibitor (as described herein), or CAR cells (as described herein); or an anti-SLAMF7 antibody (as described herein); or an antibody-drug conjugate (as described herein); or a nuclear export inhibitor (as described herein).

In some embodiments, the additional active agent used together with an inhibitor of NSD2 provided herein, in the methods and compositions described herein is dexamethasone.

In some embodiments, the dexamethasone is administered at a 4 mg dose on days 1 and 8 of a 21 day cycle. In some other embodiments, the dexamethasone is administered at a 4 mg dose on days 1, 4, 8 and 11 of a 21 day cycle. In some embodiments, the dexamethasone is administered at a 4 mg dose on days 1, 8, and 15 of a 28 day cycle. In some other embodiments, the dexamethasone is administered at a 4 mg dose on days 1, 4, 8, 11, 15 and 18 of a 28 day cycle. In some embodiments, the dexamethasone is administered at a 4 mg dose on days 1, 8, 15, and 22 of a 28 day cycle. In one such embodiment, the dexamethasone is administered at a 4 mg dose on days 1, 10, 15, and 22 of Cycle 1. In some embodiments, the dexamethasone is administered at a 4 mg dose on days 1, 3, 15, and 17 of a 28 day cycle. In one such embodiment, the dexamethasone is administered at a 4 mg dose on days 1, 3, 14, and 17 of Cycle 1.

In some other embodiments, the dexamethasone is administered at an 8 mg dose on days 1 and 8 of a 21 day cycle. In some other embodiments, the dexamethasone is administered at an 8 mg dose on days 1, 4, 8 and 11 of a 21 day cycle. In some embodiments, the dexamethasone is administered at an 8 mg dose on days 1, 8, and 15 of a 28 day cycle. In some other embodiments, the dexamethasone is administered at an 8 mg dose on days 1, 4, 8, 11, 15 and 18 of a 28 day cycle. In some embodiments, the dexamethasone is administered at an 8 mg dose on days 1, 8, 15, and 22 of a 28 day cycle. In one such embodiment, the dexamethasone is administered at an 8 mg dose on days 1, 10, 15, and 22 of Cycle 1. In some embodiments, the dexamethasone is administered at an 8 mg dose on days 1, 3, 15, and 17 of a 28 day cycle. In one such embodiment, the dexamethasone is administered at an 8 mg dose on days 1, 3, 14, and 17 of Cycle 1.

In some embodiments, the dexamethasone is administered at a 10 mg dose on days 1 and 8 of a 21 day cycle. In some other embodiments, the dexamethasone is administered at a 10 mg dose on days 1, 4, 8 and 11 of a 21 day cycle. In some embodiments, the dexamethasone is administered at a 10 mg dose on days 1, 8, and 15 of a 28 day cycle. In some other embodiments, the dexamethasone is administered at a 10 mg dose on days 1, 4, 8, 11, 15 and 18 of a 28 day cycle. In some embodiments, the dexamethasone is administered at a 10 mg dose on days 1, 8, 15, and 22 of a 28 day cycle. In some embodiments, the dexamethasone is administered at a 10 mg dose on days 1, 10, 15, and 22 of Cycle 1. In some embodiments, the dexamethasone is administered at a 10 mg dose on days 1, 3, 15, and 17 of a 28 day cycle. In some embodiments, the dexamethasone is administered at a 10 mg dose on days 1, 3, 14, and 17 of Cycle 1.

In some embodiments, the dexamethasone is administered at a 20 mg dose on days 1 and 8 of a 21 day cycle. In some other embodiments, the dexamethasone is administered at a 20 mg dose on days 1, 4, 8 and 11 of a 21 day cycle. In some embodiments, the dexamethasone is administered at a 20 mg dose on days 1, 8, and 15 of a 28 day cycle. In some other embodiments, the dexamethasone is administered at a 20 mg dose on days 1, 4, 8, 11, 15 and 18 of a 28 day cycle. In some embodiments, the dexamethasone is administered at a 20 mg dose on days 1, 8, 15, and 22 of a 28 day cycle. In some embodiments, the dexamethasone is administered at a 20 mg dose on days 1, 10, 15, and 22 of Cycle 1. In some embodiments, the dexamethasone is administered at a 20 mg dose on days 1, 3, 15, and 17 of a 28 day cycle. In some embodiments, the dexamethasone is administered at a 20 mg dose on days 1, 3, 14, and 17 of Cycle 1.

In some embodiments, the dexamethasone is administered at a 40 mg dose on days 1 and 8 of a 21 day cycle. In some other embodiments, the dexamethasone is administered at a 40 mg dose on days 1, 4, 8 and 11 of a 21 day cycle. In some embodiments, the dexamethasone is administered at a 40 mg dose on days 1, 8, and 15 of a 28 day cycle. In one such embodiment, the dexamethasone is administered at a 40 mg dose on days 1, 10, 15, and 22 of Cycle 1. In some other embodiments, the dexamethasone is administered at a 40 mg dose on days 1, 4, 8, 11, 15 and 18 of a 28 day cycle. In other such embodiments, the dexamethasone is administered at a 40 mg dose on days 1, 8, 15, and 22 of a 28 day cycle. In other such embodiments, the dexamethasone is administered at a 40 mg dose on days 1, 3, 15, and 17 of a 28 day cycle. In some embodiments, the dexamethasone is administered at a 40 mg dose on days 1, 3, 14, and 17 of Cycle 1.

In another embodiment, the additional active agent used together with an inhibitor of NSD2 provided herein in the methods and compositions described herein is bortezomib. In yet another embodiment, the additional active agent used together with an inhibitor of NSD2 provided herein in the methods and compositions described herein is daratumumab. In some such embodiments, the methods additionally comprise administration of dexamethasone. In some embodiments, the methods comprise administration of an inhibitor of NSD2 provided herein with a proteasome inhibitor as described herein, a CD38 inhibitor as described herein and a corticosteroid as described herein.

In certain embodiments, an inhibitor of NSD2 provided herein is administered in combination with checkpoint inhibitors. In some embodiments, one checkpoint inhibitor is used in combination with an inhibitor of NSD2 provided herein in connection with the methods provided herein. In another embodiment, two checkpoint inhibitors are used in combination with an inhibitor of NSD2 provided herein in connection with the methods provided herein. In yet another embodiment, three or more checkpoint inhibitors are used in combination with an inhibitor of NSD2 provided herein in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-Ll and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In some embodiments, the checkpoint inhibitor is a CTLA-4 inhibitor. In some embodiments, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In some embodiments, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In some embodiments, the checkpoint inhibitor is a PD-1/PD-L1 inhibitor. Examples of PD-l/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In some embodiments, the PD-1 inhibitor is an anti-PD-1 antibody. In some embodiments, the anti-PD-1 antibody is BGB-A317, nivolumab (also known as ONO-4538, BMS-936558, or MDX1106) or pembrolizumab (also known as MK-3475, SCH 900475, or lambrolizumab). In some embodiments, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In some embodiments, the checkpoint inhibitor is a PD-L1 inhibitor. In some embodiments, the PD-L1 inhibitor is an anti-PD-L1 antibody. In some embodiments, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In some embodiments, the checkpoint inhibitor is a PD-L2 inhibitor. In some embodiments, the PD-L2 inhibitor is an anti-PD-L2 antibody. In some embodiments, the anti-PD-L2 antibody is rHIgM12B7A.

In some embodiments, the checkpoint inhibitor is a lymphocyte activation gene-3 (LAG-3) inhibitor. In some embodiments, the LAG-3 inhibitor is IMP321, a soluble Ig fusion protein (Brignone et al., J. Immunol., 2007, 179, 4202-4211). In another embodiment, the LAG-3 inhibitor is BMS-986016.

In some embodiments, the checkpoint inhibitors is a B7 inhibitor. In some embodiments, the B7 inhibitor is a B7-H3 inhibitor or a B7-H4 inhibitor. In some embodiments, the B7-H3 inhibitor is MGA271, an anti-B7-H3 antibody (Loo et al., Clin. Cancer Res., 2012, 3834).

In some embodiments, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In some embodiments, the checkpoint inhibitor is an OX40 (CD134) agonist. In some embodiments, the checkpoint inhibitor is an anti-OX40 antibody. In some embodiments, the anti-OX40 antibody is anti-OX-40. In another embodiment, the anti-OX40 antibody is MEDI6469.

In some embodiments, the checkpoint inhibitor is a GITR agonist. In some embodiments, the checkpoint inhibitor is an anti-GITR antibody. In some embodiments, the anti-GITR antibody is TRX518.

In some embodiments, the checkpoint inhibitor is a CD137 agonist. In some embodiments, the checkpoint inhibitor is an anti-CD137 antibody. In some embodiments, the anti-CD137 antibody is urelumab. In another embodiment, the anti-CD137 antibody is PF-05082566.

In some embodiments, the checkpoint inhibitor is a CD40 agonist. In some embodiments, the checkpoint inhibitor is an anti-CD40 antibody. In some embodiments, the anti-CD40 antibody is CF-870,893.

In some embodiment, the checkpoint inhibitor is recombinant human interleukin-15 (rhIL-15).

In some embodiment, the checkpoint inhibitor is an IDO inhibitor. In some embodiments, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with one or more second therapeutic agents as described herein where appropriate for treating diseases described herein and understood in the art.

In certain embodiments, an inhibitor of NSD2 provided herein can be used in combination with one or more immune cells expressing one or more chimeric antigen receptors (CARs) on their surface (e.g., a modified immune cell). Generally, CARs comprise an extracellular domain from a first protein (e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a V_{L} linked to V_{H} by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ES0-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS.

In various specific embodiments, the tumor-associated antigen or tumor-specific antigen is an AML-related tumor antigens, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is a CD3ζ intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3ζ signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 1 05(11):4247-4254 (2005).

In certain embodiments, an inhibitor of NSD2 provided herein is administered to a subject with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

In certain embodiments, an inhibitor of NSD2 provided herein is administered to a subject with various types or stages of multiple myeloma in combination with a deacetylase inhibitor, such as a histone deacetylase inhibitor (HDAC inhibitor). Suitable DAC or HDAC inhibitors include, for example, 1) hydroxamic acid derivatives; 2) short-chain fatty acids (SCFAs); 3) cyclic tetrapeptides; 4) benzamides; 5) electrophilic ketones; and/or any other class of compounds capable of inhibiting histone deacetylase. In certain embodiments, the HDAC inhibitor is, for example, Suberoylanilide Hydroxamic Acid (SAHA) or LAQ 824. In certain embodiments, the HDAC is panobinostat. In certain embodiments, the HDAC inhibitor is panobinostat, and is used in combination with bortezomib and dexamethasone.

In certain embodiments, an inhibitor of NSD2 provided herein is administered to a subject with various types or stages of multiple myeloma in combination with an anti-SLAMF7 antibody. In certain embodiments, the anti-SLAMF7 antibody is elotuzumab. In certain embodiments, the anti-SLAMF7 antibody is elotuzumab, and is used in combination with lenalidomide and dexamethasone.

In certain embodiments, an inhibitor of NSD2 provided herein is administered to a subject with various types or stages of multiple myeloma in combination with an antibody-drug conjugate, i.e., an antibody conjugated to a conjugation moiety or an agent such as a label or toxin. A conjugation moiety can be any conjugation moiety deemed useful to one of skill in the art. For instance, a conjugation moiety can be a polymer, such as polyethylene glycol, that can improve the stability of the antibody in vitro or in vivo. A conjugation moiety can have therapeutic activity, thereby yielding an antibody-drug conjugate. A conjugation moiety can be a molecular payload that is harmful to target cells. A conjugation moiety can be a label useful for detection or diagnosis. In certain aspects, a conjugation moiety is linked to the antibody via a direct covalent bond. In certain aspects, a conjugation moiety is linked to the antibody via a linker. In particular aspects, a conjugation moiety or a linker is attached via one or more non-natural amino acids of an antibody.

In certain embodiments, an inhibitor of NSD2 provided herein is administered to a subject with various types or stages of multiple myeloma in combination with a nuclear export inhibitor, also known as a selective inhibitor of nuclear export (SINE). In certain embodiments, the nuclear export inhibitor is leptomycin B. In certain embodiments, the nuclear export inhibitor is an exportin 1 (XPO1) inhibitor, such as Selinexor (KPT-330). In certain embodiments, the nuclear export inhibitor is Selinexor and is used in combination with dexamethasone.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

### Example 1. Analysis of Newly-Diagnosed Multiple Myeloma Patient Data Sets

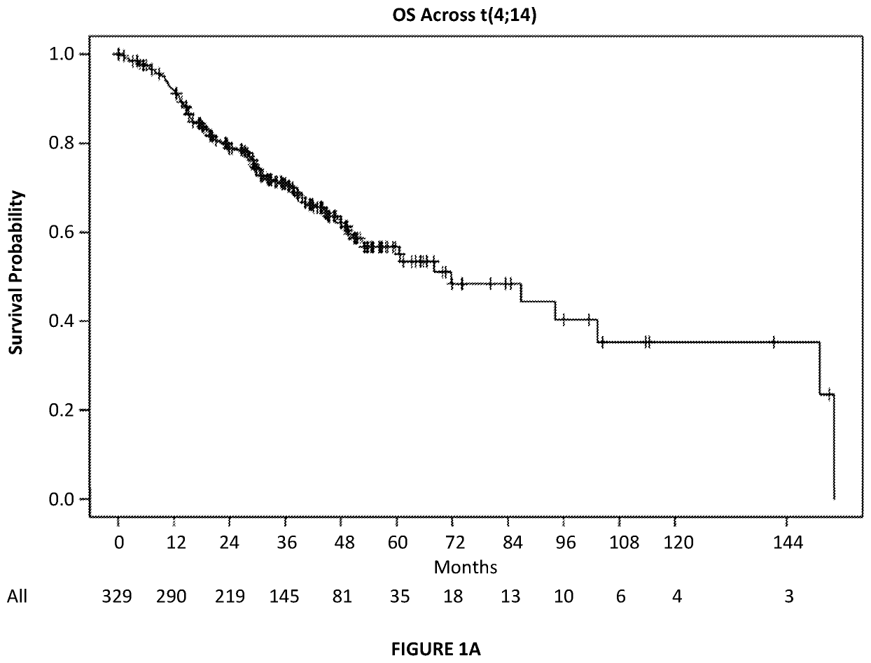

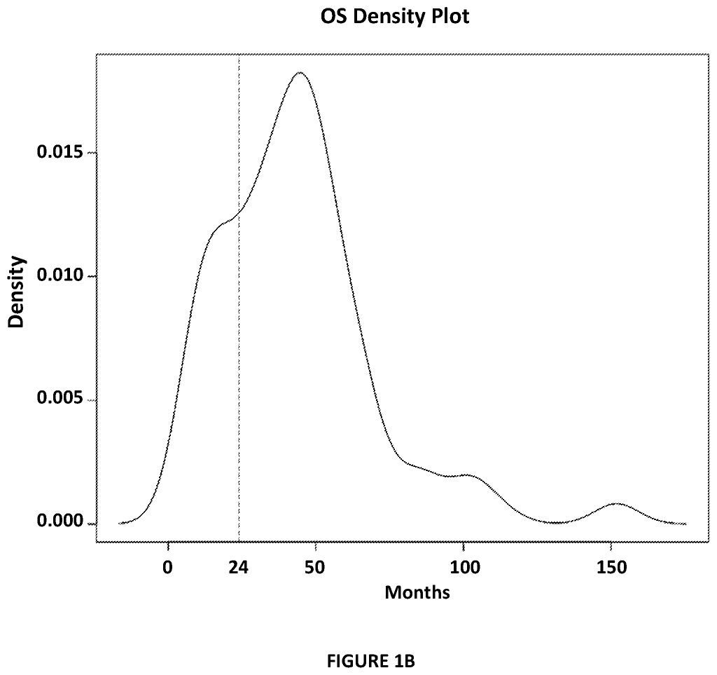

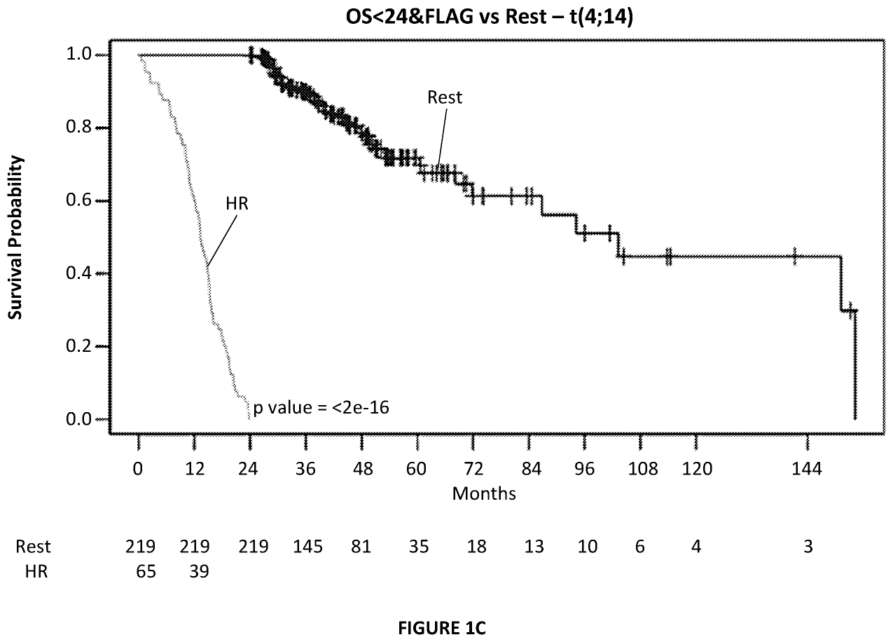

Samples from 329 newly diagnosed multiple myeloma (NDMM) patients were obtained. The samples from the 329 NDMM patients were analyzed using either whole exome sequencing or whole genome sequencing. Each of the patient samples had a previously identified T4;14 translocation. FIG. 1A shows the survival probability of the 329 NDMM patients over time.

A landmark analysis of the 329 NDMM patients using overall survival of less than 24 months was then performed. FIG. 1B shows a density plot of the overall survival of the 329 NDMM patients. A subset of high-risk patients (HR) were then identified and grouped according to patients having overall survival of less than 24 months (FIG. 1C). MANTA output was parsed to pull out translocation calls with breakpoints in 4p corresponding to the previously identified T4;14 translocation. Translocations between chromosomes 4 and 14 were pulled out into data matrices containing calls, translocation partner locations, variant allele frequencies, and the distance of the translocation from a fixed genomic point: the transcription start site of FGFR3. These data matrices were used in a supervised analysis with the defined high risk and non-high risk separation. Further genomic analysis was then performed on the subset of high-risk patient samples, which were samples from patients having overall survival of less than 24 months.

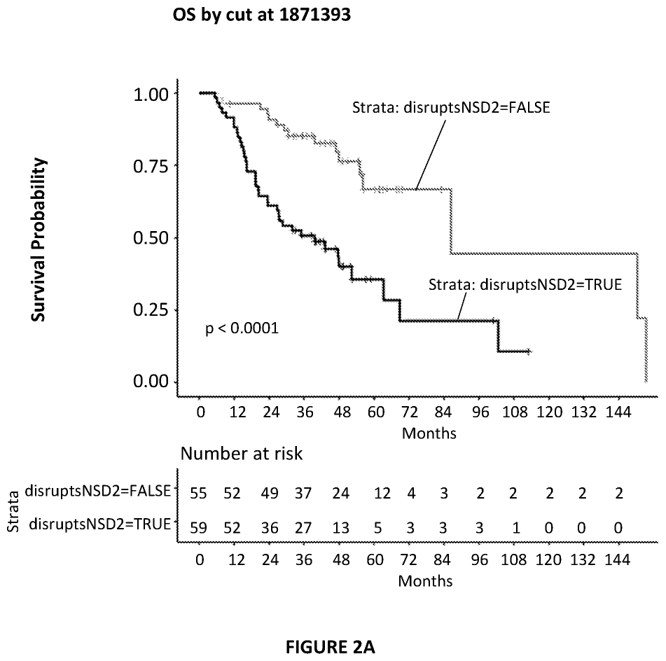

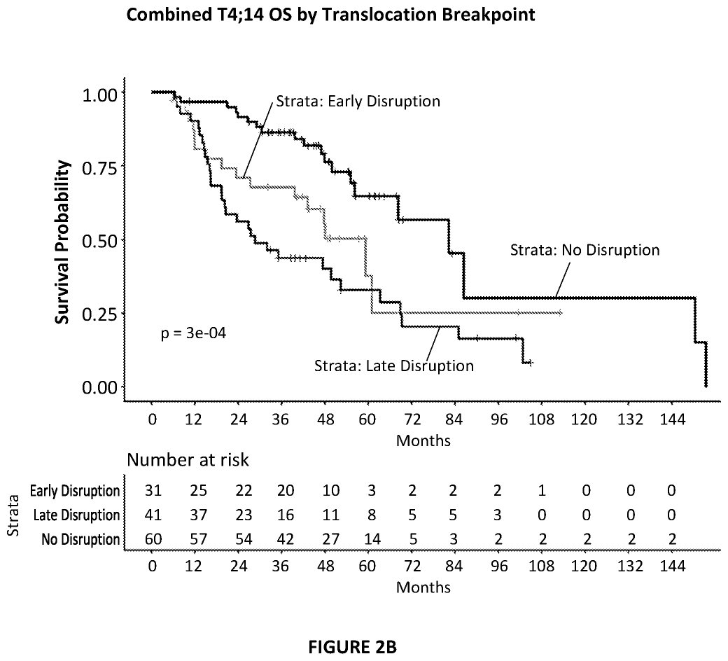

Further genomic analysis demonstrated a difference in overall survival correlated to translocations that either disrupt the coding of the Nuclear Receptor Binding SET Domain Protein 2 (NSD2) protein or do not disrupt the coding of the NSD2 protein. As demonstrated by FIG. 2A, translocations that disrupt the coding of the NSD2 protein correlate with shorter overall survival.

The patient data was then grouped in to three areas relative to the Nuclear Receptor Binding SET Domain Protein 2 (NSD2) gene: (1) upstream of the transcription start site, (2) upstream of the canonical translation start site, and (3) within the gene body disrupting the coding sequence. Kaplan-Meier plots were generated for patient groups from these 3 areas. Using these genomic features (canonical transcription and translation start site) patients were separated into three groups: no disruption, early disruption, and late disruption, with each group having an increasingly poor prognosis in terms of time to overall survival (see FIG. 2B). A no disruption was defined as a T4:14 translocation with a breakpoint between nucleotide 1,808,872 and nucleotide 1,871,393 of the forward strand, according to genomic build GRCh38.p13. An early disruption was defined as a T4:14 translocation with a disruption between nucleotide 1,871,393 and nucleotide 1,900,655 of the forward strand, according to genomic build GRCh38.p13. A late disruption was defined as a T4:14 translocation with a breakpoint in the NSD2 gene after nucleotide 1,900,655 of the forward strand, according to genomic build GRCh38.p13. This example demonstrates that disruption of the NSD2 gene correlates with reduced overall survival for NDMM patients having T4:14 translocations.

### Example 2. Analysis of Patient RNA-SEQ Data

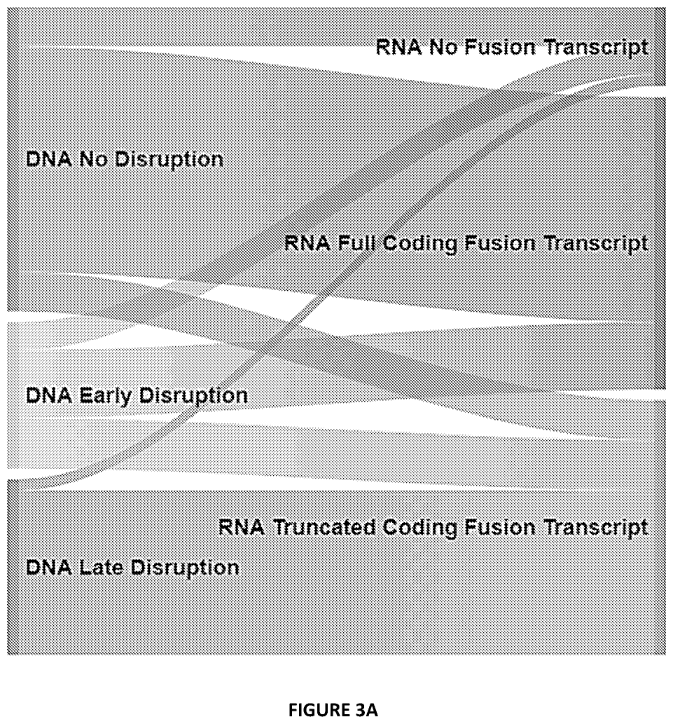

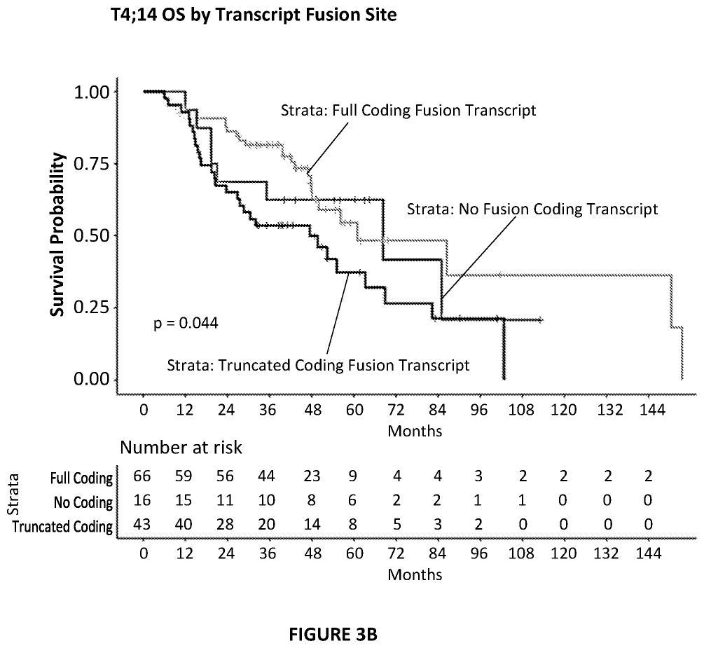

Fusion transcripts result from chromosomal rearrangements and are drivers in certain cancers, including Multiple Myeloma. NDMM patient RNA-seq data was analyzed with the STAR-fusion pipeline to identify expressed fusion transcripts. Fusions were found to occur with the first NSD2 coding exon, generating a full coding transcript, or to a later NSD2 coding exon, generating a truncated transcript. Analysis of the RNA-seq data identified three groups of patients: (1) the full coding fusion transcript group, where there is no disruption of the protein coding sequence or translated protein, (2) the truncated fusion transcript group characterized by late DNA disruption, and (3) a small subset of patients having no measured fusion transcript expression (See FIG. 3A). Kaplan-Meier plots were generated for these three patient groups based on expression of the fusion transcript. The patients expressing the truncated fusion protein have a worse overall survival than patients expressing the full coding fusion transcript or no fusion transcript (see FIG. 3B).

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

What is claimed is:

1. A method of treating multiple myeloma, comprising administering to a subject with multiple myeloma a therapeutically effective amount of an inhibitor of Nuclear Receptor Binding SET Domain Protein 2 (NSD2).

2. The method of claim 1, wherein the multiple myeloma has previously been determined to have a 4:14 chromosome translocation (t(4;14)).

3. The method of claim 2, wherein the t(4;14) results in a disruption in the NSD2 gene.

4. The method of claim 3, wherein the disruption in the NSD2 gene is located after the transcription start site of NSD2.

5. The method of claim 3 or claim 4, wherein the disruption in the NSD2 gene is located after the translation start site of NSD2.

6. The method of any one of claims 3-5, wherein the disruption in the NSD2 gene is located before the translation stop site of NSD2.

7. The method of any one of claims 3-5, wherein the disruption in the NSD2 gene is located before the first coding exon of the NSD2 gene.

8. The method of any one of claims 3-6, wherein the disruption in the NSD2 gene is located in the first coding exon of the NSD2 gene.

9. The method of any one of claims 3-6, wherein the disruption in the NSD2 gene is located between the start of the first coding exon and the start of the second coding exon of the NSD2 gene.

10. The method of any one of claims 3-9, wherein the disruption in the NSD2 gene is located at or after genomic position 1,871,393 of Genome Reference Consortium Human Build 38 patch release 13 (GRCh38.p13).

11. The method of any one of claims 3-10, wherein the disruption in the NSD2 gene is located between genomic position 1,871,393 and genomic position 1,900,655 of GRCh38.p13.

12. The method of any one of claims 3-10, wherein the disruption in the NSD2 gene is located at or after genomic position 1,900,655 of GRCh38.p13.

13. The method of any one of claims 3-10, wherein the t(4;14) is located between genomic position 1,900,655 and genomic position 1,982,207 of GRCh38.p13.

14. The method of any one of the preceding claims, wherein the multiple myeloma expresses a truncated NSD2 protein.

15. The method of any one of claims 1-13, wherein the multiple myeloma expresses a full-length NSD2 protein.

16. The method of claim 15, wherein the multiple myeloma expresses elevated levels of the full-length NSD2 protein.

17. The method of any one of claims 2-16, wherein the 4:14 chromosome translocation (t(4;14)) was identified by a method comprising in situ hybridization, PCR, RT-PCR, RNA sequencing, fluorescence in situ hybridization (FISH), transcript in situ hybridization, whole genome sequencing, whole exome sequencing, mixed ligation probe assays, mass spectrometry, and/or MALDI-TOF.

18. The method of any one of the previous claims, wherein the inhibitor of NSD2 is selected from an antibody, a small molecule, an aptamer, an siRNA, and an antisense oligonucleotide.

19. The method of any one of the previous claims, wherein the method comprises administering at least one second therapeutic agent.

20. The method of claim 19, wherein at least one second therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, and a nuclear export inhibitor.

21. The method of claim 19, wherein at least one second therapeutic agent is selected from lenalidomide, thalidomide, pomalidomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

22. The method of claim 19, wherein the at least one second therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl)piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.

23. A method of selecting a subject with multiple myeloma for treatment with an NSD2 inhibitor, comprising determining whether the subject has a 4:14 chromosome translocation (t(4;14)), wherein if the subject has a t(4;14), the subject is selected for treatment with an NSD2 inhibitor.

24. A method of predicting whether a subject with multiple myeloma will benefit from treatment with an NSD2 inhibitor, comprising determining whether the subject has a 4:14 chromosome translocation (t(4;14)), wherein if the subject has a t(4;14) translocation, the subject is predicted to benefit from treatment with an NSD2 inhibitor.

25. The method of claim 23 or claim 24, wherein the t(4;14) results in a disruption in the NSD2 gene.

26. The method of claim 25, wherein the disruption in the NSD2 gene is located after the transcription start site of NSD2.

27. The method of claim 25 or claim 26, wherein the disruption in the NSD2 gene is located after the translation start site of NSD2.

28. The method of any one of claims 25-27, wherein the disruption in the NSD2 gene is located before the translation stop site of NSD2.

29. The method of any one of claims 25-28, wherein the disruption in the NSD2 gene is located before the first coding exon of the NSD2 gene.

30. The method of any one of claims 25-28, wherein the disruption in the NSD2 gene is located in the first coding exon of the NSD2 gene.

31. The method of any one of claims 25-28, wherein the disruption in the NSD2 gene is located between the start of the first coding exon and the start of the second coding exon of the NSD2 gene.

32. The method of any one of claims 25-31, wherein the disruption in the NSD2 gene is located at or after genomic position 1,871,393 of Genome Reference Consortium Human Build 38 patch release 13 (GRCh38.p13).

33. The method of any one of claims 25-32, wherein the disruption in the NSD2 gene is located between genomic position 1,871,393 and genomic position 1,900,655 of GRCh38.p13.

34. The method of any one of claims 25-32, wherein the disruption in the NSD2 gene is located at or after genomic position 1,900,655 of GRCh38.p13.

35. The method of any one of claims 25-32, wherein the t(4;14) is located between genomic position 1,900,655 and genomic position 1,982,207 of GRCh38.p13.

36. The method of any one of claims 23-35, wherein the multiple myeloma expresses a truncated NSD2 protein.

37. The method of any one of claims 23-35, wherein the multiple myeloma expresses a full-length NSD2 protein.

38. The method of claim 37, wherein the multiple myeloma expresses elevated levels of the full-length NSD2 protein.

39. The method of any one of claims 23-38, wherein determining whether the subject has a t(4;14) comprises in situ hybridization, PCR, RT-PCR, RNA sequencing, fluorescence in situ hybridization (FISH), transcript in situ hybridization, whole genome sequencing, whole exome sequencing, mixed ligation probe assays, mass spectrometry, and/or MALDI-TOF.

40. The method of any one of claims 23-39, wherein the method further comprises administering an NSD2 inhibitor.

41. The method of claim 40, wherein the inhibitor of NSD2 is selected from an antibody, small molecule, an aptamer, an siRNA, and an antisense oligonucleotide.

42. The method of claim 40 or claim 41, wherein the method comprises administering at least one second therapeutic agent.

43. The method of claim 42, wherein at least one second therapeutic agent is selected from a chemotherapy agent, a steroid, an immunomodulating agent, a proteasome inhibitor, a histone deacetylase inhibitor, an anti-CD38 antibody, an anti-SLAMF7 antibody, an antibody-drug conjugate, and a nuclear export inhibitor.

44. The method of claim 42, wherein at least one second therapeutic agent is selected from lenalidomide, thalidomide, pomalidomide, bortezomib, carfilzomib, ixazomib, panobinostat, melphalan, vincristine, cyclophosphamide, etoposide, doxorubicin, and bendamustine, dexamethasone, prednisone, daratumumab, isatuximab, elotuzumab, belantamab mafodotin-blmf, Selinexor, pamidronate, zoledronic acid, and denosumab.

45. The method of claim 42, wherein the at least one second therapeutic agent is selected from:
a) lenalidomide;
b) iberdomide;
c) (S)-4-(4-(4-(((2-(2,6-dioxopiperidin-3-yl)-1-oxoisoindolin-4-yl)oxy)methyl)benzyl)piperazin-1-yl)-3-fluorobenzonitrile;
d) (i) lenalidomide, pomalidomide, or thalidomide; and (ii) dexamethasone;
e) (i) carfilzomib, ixazomib, or bortezomib; (ii) lenalidomide; and (iii) dexamethasone;
f) (i) bortezomib or carfilzomib; (ii) cyclophosphamide; and (iii) dexamethasone;
g) (i) elotuzumab or daratumumab; (ii) lenalidomide; and (iii) dexamethasone;
h) bortezomib, liposomal doxorubicin, and dexamethasone;
i) panobinostat, bortezomib, and dexamethasone;
j) elotuzumab, bortezomib, and dexamethasone;
k) melphalan and prednisone, with or without thalidomide or bortezomib;
l) vincristine, doxorubicin, and dexamethasone;
m) dexamethasone, cyclophosphamide, etoposide, and cisplatin; and
n) dexamethasone, thalidomide, cisplatin, doxorubicin, cyclophosphamide, and etoposide, with or without bortezomib.
